(19) United States
(12) Patent Application Publication
(10) Pub. No.: US 2010/0256157 A1
(43) Pub. Date: Oct. 7, 2010

(54) METHOD OF TREATMENT

(76) Inventors: Jakob Busch-Petersen, King of Prussia, PA (US); Thomas B. LEONARD, Ridgefield, CT (US); Michael R. Palovich, King Of Prussia, PA (US); Henry M. Sarau, Green Lane, PA (US); Aili L. Lazaar, King of Prussia, PA (US); Katherine L. Widdowson, Collegeville, PA (US)

Correspondence Address: GlaxoSmithKline;GLOBAL PATENTS -US, UW2220, P. O. BOX 1539, KING OF PRUSSIA, PA, 19406-0939, US

(21) Appl. No.: 12/727,488

(22) Filed: Mar. 19, 2010

Related U.S. Application Data
(63) Continuation-in-part of application No. PCT/US2008/076519, filed on Sep. 16, 2008.
Application No. PCT/US2008/076519 is a continuation-in-part of application No. 11/869,055, filed on Oct. 9, 2007, now Pat. No. 7,709,485.
Application No. 11/869,055 is a continuation of application No. 10/532,956, filed on Apr. 27, 2005, now abandoned.
(60) Provisional application No. 60/974,175, filed on Sep. 21, 2007.
(60) Provisional application No. 60/421,956, filed on Oct. 29, 2002.

(51) Int. Cl. A61K 31/495; A61P 1/18; A61P 11/00

(52) U.S. Cl. 514/252.12

(57) ABSTRACT

This invention relates to the use of a sulfonamide substituted diphenyl urea compound to treat cystic fibrosis, or the symptoms associated with cystic fibrosis.

### CROSS REFERENCE TO PRIOR APPLICATIONS

This application is a continuation in part of International Application No. PCT/US2008/076519 filed 16 Sep. 2008, which claims the benefit of U.S. Ser. No. 60/974,175 filed 21 Sep. 2007; and of U.S. application Ser. No. 11/869,055 filed 9 Oct. 2007 (allowed) which is a continuation application of U.S. Ser. No. 10/532,956 filed 27 Apr. 2005 (abandoned) which is a §371 of PCT/US2003/033964 filed 28 Oct. 2003 and which claims the benefit of U.S. Ser. No. 60/421,956 filed 29 Oct. 2002 which are all incorporated in their entirety.

### FIELD OF THE INVENTION

This invention relates to the use of sulfonamide substituted diphenyl urea compounds to treat cystic fibrosis or non-cystic fibrosis bronchiestasis.

### BACKGROUND OF THE INVENTION

CXCR2 is a well-characterized G-protein coupled receptor for a number of chemokines that share the Glu-Leu-Arg motif including interleukin-8 (IL-8, CXCL8) and growth regulated oncogene alpha, beta and gamma, (GROα,β,γ or CXCL1,2,3) that are known to be involved in the recruitment of neutrophils to a site of injury [Reutershan, J. (2004) Drug News Perspect 19:615-623]. CXCR2 is expressed primarily on neutrophils (PMN), but can be expressed on other leukocytes as well such as monocytes. Antagonism of CXCR2 has been shown to be effective in blocking the recruitment of PMN to the lung in response to stimuli such as LPS, cigarette smoke, or ozone exposure. It is expected that antagonism of CXCR2 would be of utility in preventing the recruitment of neutrophils to the lung in response to a wide spectrum of conditions related to disease, such as cystic fibrosis or non-cystic fibrosis bronchiectasis.

Cystic fibrosis (CF) is a disease primarily characterized by recurrent cycles of infection and inflammation, particularly neutrophil predominant pulmonary cellular infiltration. It is the most common genetic disease in Caucasians. Cystic fibrosis is a disease involving epithelial cells throughout the body, causing mucus in the body to become thick, dry and sticky. Symptoms include blocked intestines at birth, salty sweat or skin, diarrhea, breathing problems, lung infection, persistent cough, wheezing, clubbing of the fingers, rectal prolapse and polyps in the nose or sinuses. Non-CF bronchiectasis (bronchiectasis of the lung caused by conditions other than cystic fibrosis) is characterized by cough, sputum production, recurrent infection and exacerbation. The lung inflammation and destruction in non-CF bronchiectasis is caused in-part by exuberant neutrophilic inflammation. A CXCR2 inhibitor would thus be expected to decrease neutrophilic damage to the lung, decrease inflammation, and thus, preserve lung function and diminish symptoms associated with the disease.

In people with cystic fibrosis, buildup of thick mucus obstructs the airways, making breathing difficult and creating an environment for infection-causing bacteria to grow. To fight infection, neutrophils migrate to the lungs, releasing enzymes such as elastase which can damage the lungs. Neutrophils also release DNA and participate in killing of bacteria, which further thickens the mucus. The thicker mucus, in turn, causes more obstruction, which causes more infection and inflammation. The natural history of cystic fibrosis is such that there is an inexorable progression of disease symptoms resulting in continued loss of pulmonary function with increasing morbidity and mortality.

Current therapeutic goals are aimed at improving airway clearance and treating infections. Treatments for cystic fibrosis lung disease presently include inhaled hypertonic saline and DNAse to aid in airway clearance, as well inhaled antibiotics, and anti-inflammatory agents such as inhaled corticosteroids or oral azithromycin. Other agents, such as high dose ibuprofen or oral steroids, which are effective anti-inflammatory agents, have their use limited due to their gastrointestinal effects and adverse effects on bone density.

There is considerable evidence to suggest that the recruitment and activation of inflammatory cells in the lung contributes significantly to the pathophysiology of cystic fibrosis. It has been proposed that a CXCR2 receptor antagonist would specifically inhibit the recruitment and activation of neutrophils into the lung, and thus would significantly help to slow disease progression, deliver rapid improvement in lung function and/or symptoms.

Antagonism of the CXCR2 receptor has the potential to affect several aspects of cystic fibrosis. CXCR2 agonists such as IL-8/CXCL8 and ENA-78/CXCL5 are highly expressed in the airways of patients with cystic fibrosis. IL-8 is considered to play a pivotal role in chronic airway inflammation through its actions as a neutrophil chemoattractant. The recruitment of activated neutrophils into the lung results in release of chromatin as well as proteases and oxidants. In addition, neutrophil killing of bacteria can release DNA. IL-8 is also elevated in the serum of patients with cystic fibrosis, where it may be involved in systemic processes such as osteoporosis or weight loss.

CXCR1 and CXCR2 expression is increased on peripheral blood neutrophils in patients with cystic fibrosis. Sputum concentrations of IL-8, MPO, and DNA appear to correlate with pulmonary function in subjects with cystic fibrosis. CXCR2 is also expressed on other cell types within the lung such as airway smooth muscle cells. CXCR2 has been shown to mediate smooth muscle cell contractile responses, suggesting a possible role for the receptor in regulating airway hyperreactivity.

The dysregulated expression of IL-8 and CXCR2 in patients with cystic fibrosis suggests that inhibition of CXCR2 may provide a therapeutic benefit to patients with cystic fibrosis.

### SUMMARY OF THE INVENTION

The present invention relates to a method of treating cystic fibrosis in a human subject, previously diagnosed as having cystic fibrosis, in need thereof. The method involves administering to the subject having cystic fibrosis, N-[4-chloro-2-hydroxy-3-(piperazine-1-sulfonyl)phenyl]-N′-(2-chloro-3-fluorophenyl)urea, or a pharmaceutically acceptable salt thereof, in an amount effective to treat cystic fibrosis, or symptoms, such as reduced lung function, cough, exacerbations and mucus accumulation in the lung. associated with cystic fibrosis in that subject.

The present invention also relates to a method of slowing the progression of cystic fibrosis in a human subject in need thereof. The method involves administering to the subject N-[4-chloro-2-hydroxy-3-(piperazine-1-sulfonyl)phenyl]-N′-(2-chloro-3-fluorophenyl)urea, or a pharmaceutically acceptable salt thereof, in an amount effective to slow the progression of cystic fibrosis, or the symptoms of cystic fibrosis in the subject. The present invention also relates to a method of alleviating the symptoms of cystic fibrosis in a human subject in need thereof. The method involves administering to the subject N-[4-chloro-2-hydroxy-3-(piperazine-1-sulfonyl)phenyl]-N′-(2-chloro-3-fluorophenyl)urea, or a pharmaceutically acceptable salt thereof, in an amount effective to alleviate the symptoms of cystic fibrosis in the subject.

The present invention also relates to a method of treating the inflammatory response, such as increased lung neutrophilia or mucus hypersecretion, associated with cystic fibrosis in a patient who has been diagnosed with cystic fibrosis. This method involves administering to the subject N-[4-chloro-2-hydroxy-3-(piperazine-1-sulfonyl)phenyl]-N′-(2-chloro-3-fluorophenyl)urea, or a pharmaceutically acceptable salt thereof, in an amount effective to decrease the inflammation associated with cystic fibrosis in the subject.

### DETAILED DESCRIPTION OF THE INVENTION

The present invention is directed to various methods of treating cystic fibrosis, or the symptoms associated with cystic fibrosis in a mammal, suitably a human in need thereof, by administering to said mammal or human, an effective amount of N-[4-chloro-2-hydroxy-3-(piperazine-1-sulfonyl)phenyl]-N′-(2-chloro-3-fluorophenyl)urea or a pharmaceutically acceptable salt thereof. For purposes herein, successful treatment of cystic fibrosis encompasses, but is not limited to, reducing inflammation in the lung present in a subject, reducing mucus accumulation in the lung, reducing excessive mucus production, reducing pulmonary neutrophilia, reducing mucus production generally, decreasing mucus dysfunction, reducing mucus viscosity leading to improved mucociliary clearance, improving lung function, reducing risk of exacerbations, diminishing cough, reducing the frequency and/or severity of exacerbations which can include hospitalizations, thereby reducing the number of hospitalizations for a subject have cystic fibrosis.

Another embodiment of the invention is directed to a method of slowing the progression of cystic fibrosis in a mammal, suitably a human in need thereof, which comprises administering to said mammal, or human, an effective amount of N-[4-chloro-2-hydroxy-3-(piperazine-1-sulfonyl)phenyl]-N′-(2-chloro-3-fluorophenyl)urea, or a pharmaceutically acceptable salt thereof. For purposes herein, successful treatment of slowing the progression of cystic fibrosis encompasses, but is not limited to, reducing pulmonary neutrophilia, reducing mucus production, decreasing mucus dysfunction, reducing mucus viscosity leading to improved mucocilliary clearance, improving lung function, reducing the risk of exacerbations, diminishing cough., reducing frequency and/or severity of exacerbations, which can include hospitalizations, thereby reducing the number of hospitalizations for a subject have cystic fibrosis.

This invention also provides for a method of alleviating or reducing the symptoms of cystic fibrosis which comprises administering to a mammal, suitably a human in need thereof, an effective amount of N-[4-chloro-2-hydroxy-3-(piperazine-1-sulfonyl)phenyl]-N′-(2-chloro-3-fluorophenyl)urea, or a pharmaceutically acceptable salt thereof. For the purposes herein, symptoms of cystic fibrosis include, but are not limited to, breathing problems, lung infection, persistent cough, wheezing, excessive mucus production and clubbing of the fingers. Therefore successful treatment with this selective CXCR2 antagonist is expected to improve lung function, reduce the number and severity of infection induced exacerbations (and associated hospitalizations) and improve patient's quality of life ultimately resulting in increased survival.

Airway neutrophilia has been observed in infants with CF, and increases throughout life, is associated with decreased lung function, thickened mucus, reduced microbial clearance, and protease-mediated lung destruction. Therefore, another aspect of this invention is the treatment of pediatrics and infants diagnosed with cystic fibrosis.

The compound, N-[4-chloro-2-hydroxy-3-(piperazine-1-sulfonyl)phenyl]-N′-(2-chloro-3-fluorophenyl)urea, and/or its pharmaceutically acceptable salts, offer an alternative for the treatment of cystic fibrosis, representing a different class of compounds to currently available treatment options. As noted above, the mainstay of initial therapy for cystic fibrosis includes inhaled hypertonic saline and DNAse to aid in airway clearance. Inhaled antibiotics and anti-inflammatory agents are also utilized. Patients with cystic fibrosis are currently treated with several classes of therapeutic agents which, in some patients, are either ineffective or have significant side effects. The present compound offers an alternative treatment option which may provide a better benefit/risk ratio to the patient.

According to the present invention, a subject diagnosed as having cystic fibrosis such as one who has been genetically identified as an asymptomatic individual, or a patient identified by a salty sweat test, or is a patient identified by CT as having bronchiectasis but who is asymptomatic may be treated with an effective amount of N-[4-chloro-2-hydroxy-3-(piperazine-1-sulfonyl)phenyl]-N′-(2-chloro-3-fluorophenyl)urea, or a pharmaceutically acceptable salt thereof

Another aspect of the invention is the use of N-[4-chloro-2-hydroxy-3-(piperazine-1-sulfonyl)phenyl]-N′-(2-chloro-3-fluorophenyl)urea, or a pharmaceutically acceptable salt thereof in the treatment of non-cystic fibrosis bronchiestasis.

The compound used in the method of the invention can be administered as a free base or as a pharmaceutically acceptable salt thereof. Suitable pharmaceutically acceptable salts are well known to those skilled in the art, and include salts of inorganic and organic acids, such as hydrochloric acid, hydrobromic acid, sulfuric acid, phosphoric acid, methane sulfonic acid, ethane sulfonic acid, toluenesulfonic acid, acetic acid, malic acid, tartaric acid, citric acid, lactic acid, oxalic acid, succinic acid, fumaric acid, maleic acid, benzoic acid, salicylic acid, phenylacetic acid and mandelic acid.

Pharmaceutically acceptable salts may also be formed with pharmaceutically acceptable cations. Suitable pharmaceutically acceptable cations are well known to those skilled in the art and include alkaline, alkaline earth, ammonium and quaternary ammonium cations.

One embodiment of the invention is the hydrochloric acid salt of N-[4-chloro-2-hydroxy-3-(piperazine-1-sulfonyl)phenyl]-N′-(2-chloro-3-fluorophenyl)urea.

Another embodiment of the invention is the p-toluenesulfonic acid salt of N-[4-chloro-2-hydroxy-3-(piperazine-1-sulfonyl)phenyl]-N′-(2-chloro-3-fluorophenyl)urea.

For purposes herein the term “compound” refers to N-[4-chloro-2-hydroxy-3-(piperazine-1-sulfonyl)phenyl]-N′-(2-chloro-3-fluorophenyl)urea and any pharmaceutically acceptable salt thereof of said urea.

In order to use the present compound in therapy, it will normally be formulated into a pharmaceutical composition in accordance with standard pharmaceutical practice. This invention, therefore, also relates to use of a pharmaceutical composition comprising an effective, amount of N-[4-chloro-2-hydroxy-3-(piperazine-1-sulfonyl)phenyl]-N′-(2-chloro-3-fluorophenyl)urea or a pharmaceutically acceptable salt thereof, and at least one pharmaceutically acceptable carrier or diluent in any the methods described herein.

The present compound and a pharmaceutical composition incorporating said compound, or salt thereof may conveniently be administered by any of the routes conventionally used for drug administration, for instance orally, parenterally, or via inhalation. The present compounds may also be administered in conventional dosages in combination with other known, second therapeutically active compounds which are also used for the treatment of cystic fibrosis, or symptoms associated with a patient being diagnosed with cystic fibrosis. The present compound may be administered in conventional dosage forms prepared by combining with standard pharmaceutical carriers according to conventional procedures. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof.

The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time delay material well known to the art, such as glyceryl mono-stearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1 g. When a liquid carrier is used, the preparation will be in the form of a syrup, emulsion, soft gelatin capsule, sterile injectable liquids or nonaqueous liquid suspensions.

The present compound may be employed via suitable methods of administration, including oral, parenteral, intravenous, intramuscular, subcutaneous, intranasal and intraperitoneal administration. Appropriate dosage forms for such administration may be prepared by conventional techniques.

For all methods of use disclosed herein regardless of age, the daily oral dosage regimen will preferably be from about 0.1 to about 3 mg/kg of total body weight given in divided doses if necessary The daily parenteral dosage regimen about 0.001 to about 3 mg/kg of total body weight. A once daily oral dosage is preferable. It is expected that for an adult, the amount dosed will be from about 20 mg to about 150 mg. In one embodiment the amount is from about 20-100 mg/person. Although, it will also be recognized by one of skill in the art that the optimal quantity and spacing of individual dosages of a present compound will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a present compound of or a pharmaceutically acceptable salt thereof given per day for a defined number of days, can be ascertained by those skilled in the art using conventional course of treatment determination tests.

The invention will now be described by reference to the following biological examples, which are merely illustrative and are not to be construed as a limitation of the scope of the present invention.

### Biological Examples

The compound used in the methods of the present invention may be tested in the following assays.

### Receptor Binding Assay:

[¹²⁵] IL-8 (human recombinant, IM249) was obtained from Amersham Corp., Arlington Heights, Ill., with specific activity 2000 Ci/mmol. All other chemicals were of analytical grade.

High levels of recombinant human CXCR1 and CXCR2 receptors were individually expressed in Chinese hamster ovary cells as described in Holmes, et al., Science, 1991, 253, 1278, incorporated herein to the extent required to perform the present assay. The Chinese hamster ovary membranes were prepared according to Haour, et al., J. Biol. Chem., 249 pp 2195-2205 (1974), incorporated herein to the extent required to perform the present assay, except that the homogenization buffer was changed to 40 mM Tris-HCL pH 7.5 containing 1 mM MgS0₄, 0.5 mM EDTA (ethylene-diaminetetra-acetic acid), 1 mM PMSF (α-toluene-sulphonyl fluoride), 2.5 mg/L Leupeptin and 0.1 mg/ml Aprotinin. Membrane protein concentration was determined using Bio-Rad Reagent using bovine serum albumin as a standard. All binding assays were conducted using Scintillation Proximity Assay (SPA assay) using wheatgerm agglutinin beads in a 96-well micro plate (optiplate 96, Packard) format. The membranes, CHO—CXCR1 or CHO—CXCR2, were pre-incubated with the beads in binding buffer; 20 mM Bis Tris propane, pH 8 containing 25 mM NaCl, 1 mM MgSO₄, 0.1 mM EDTA at 4° C. for 30 min prior to assay. The compound was diluted in 100% DMSO at 20 times the final concentration (final 1 nM to 1000 nM and 5% DMSO). The assay was performed in 0.1 ml reaction buffer containing binding buffer, membranes pre-treated with wheatgerm agglutinin beads, various concentrations of compound, 5% DMSO, 0.04% CHAP, 0.0025% BSA and 0.225 nM [¹²⁵I] IL-8. The 96-well plates were incubated on a shaking platform for 1 hour. At the end of the incubation the plates were spun for 5 min at 2000 RPM, and counted in a Top Count counter.

The recombinant CXCR1 receptor is also referred to herein as the non-permissive receptor and the recombinant CXCR2 receptor is referred to as the permissive receptor.

A compound demonstrating an IC₅₀ value of <10 uM is considered active in the present assay. The compound of Example 1, the hydrochloride salt, demonstrated an IC₅₀ of about 13 nM in this assay.

### Animal Model and Ex Vivo Studies:

There is considerable evidence to suggest that the recruitment and activation of inflammatory cells in the lung contributes significantly to the pathophysiology of cystic fibrosis. Based on pre-clinical evidence, and clinical evidence with the Pharmacopeia/Schering CXCR1/2 compound SCH527123 in smokers and HVT challenged with ozone, inhibition and recruitment and activation of the neutrophilic cells in the lung, are postulated to play a key role in the pathophysiology of cystic fibrosis

A CXCR2 antagonist represents a different class of agents as compared with those presently used in cystic fibrosis and are expected to slow the disease progression of cystic fibrosis, at least in part by breaking the cycle of neutrophilic inflammation and infection. Several animal models demonstrate inhibition of neutrophil recruitment by a CXCR2 antagonist. A relationship between inhibition of up-regulation of CD11b on neutrophils and inhibition of neutrophil recruitment has also been shown in animal model studies. The compound herein was shown to be active in in-vivo rodent models of a) pulmonary inflammation including endotoxin (LPS); b) cigarette smoke-induced pulmonary neutrophilia; and c) ozone-induced pulmonary neutrophilia. These in vivo rodent models are described below.

Balb/c mice (20-25 g) or Lewis rats (250-275 g) from the same breeding groups were received from Jackson labs and used in these studies. Animals were housed for a minimum of five days under quarantine prior to the initiation of the studies.

### a) LPS-Induced Lung Neutrophilia in Rats

For the rat LPS neutrophilia model, the compound of Example 1, e.g. the hydrochloride salt, was weighed-out and placed into a glass homogenizer with the appropriate amount of PEG-400 to yield a final concentration of 10%. The compound was homogenized in the PEG-400, and then brought to the final volume with distilled H₂O. A stock solution of 10% volume/volume of PEG-400 was prepared for further compound dilution and to yield the final vehicle. All dosing solutions were vortexed immediately prior to administration.

Male Lewis rats (325-350 g) (Charles River Labs, St. Constance, Canada), were dosed orally with either vehicle or the compound of Example 1, one hour prior to lipopolysaccharide (LPS) exposure. Rats were placed in groups of 5 into an aerosol exposure box (14×10×9 inches, Rubbermaid® 0100-2). After the compound of Example 1 was administered, animals were monitored for safe recovery before being returned to a cage. One hour post-dosing rats were exposed for 15 minutes to LPS (100 ug/ml) which was nebulized with a Hospitak nebulizer at a flow rate of 5 L/min into the exposure box. Four hours post-LPS exposure, the rats were euthanized with pentobarbital (100 mg/kg, i.p.). Standard surgical techniques were used to expose and cannulate the trachea with a 14 GA steel cannula.

Bronchoalveolar lavage (BAL) samples were collected by flushing the airways with 5 washes of 5 ml DPBS (w/o C⁺⁻ or Mg⁺⁺). To facilitate cell retrieval, the chest was gently compressed three times between each flush. The BAL wash retrieved from each rat was centrifuged at 3000×g for 10 min to pellet the cells, the supernatant was removed, and the resulting pellet was re-suspended in 5 ml saline. Total cell counts were performed manually on a hemocytometer after diluting the resuspended cells 1:2 with Tuerke solution. Differential cell slides were made with re-suspended cells using a Cytospin (5 min, 300 RPM) to form a monolayer of cells on the slide surface. The cells were stained with methylene blue and eosin (DiffQuik) and then differential counts were performed by light microscopy. A minimum of 200 cells per slide were counted.

The compound of Example 1 demonstrated dose dependent inhibition of LPS-induced neutrophil recruitment in the lungs as measured in BAL fluid. LPS exposure of rats caused a characteristic neutrophil influx into the lungs that was assessed by enumerating cell numbers from bronchoalveolar lavage fluid. Oral administration of the compound of Example 1 (0.3-30 mg/kg in 10% PEG-400) to rats caused a dose-related inhibition of BAL neutrophilia compared to vehicle-treated rats. The vehicle-treated group had a mean of 4.97±0.5×10⁶ neutrophils and a significant inhibition in BAL neutrophilia was observed at doses of 3, 10 and 30 mg/kg of the compound of Example 1. At 3 mg/kg there was 44% inhibition of neutrophils with a mean of 2.78±0.39×10⁶ cells, (p<0.05), at 10 mg/kg there was 88% inhibition (0.60±0.10×10⁶, cells, p<0.001) and at the 30 mg/kg dose 96% inhibition (0.18±0.05×10⁶cells, p<0.001). The dose required to inhibit 50% of the LPS-induced response was 3.5 mg/kg. In parallel studies, the ex vivo CXCL2-induced upregulation CD11b on neutrophils in rat serum was assessed. Following oral dosing of the compound of Example 1, or Example 2, at 10 mg/kg (i.e. either the hydrochloride or the tosylate salt), whole blood was drawn by cardiac puncture one hour after dosing in a 3-ml syringe containing 100 ul of 0.25 M EDTA (GIBCO, Grand Island, N.Y.) and filling to 3 ml. Rat CXCL2 (PeproTech, Rocky Hill, N.J.) stock was made by reconstitution in either DPBS/0.1% BSA or sterile water at 10 or 20 uM. The stock was diluted to “11×” the maximum used concentration in DPBS (GIBCO) and serially diluted in either DPBS or the diluted Kreb's/BSA vehicle. 10 ul of appropriate concentration of CXCL2 or appropriate vehicle was added to 12×75 polypropylene tubes followed by 100 ul of whole blood. Following gentle hand-agitation, the tubes were incubated for 20-30 minutes in a 37° bath, with further gentle agitation every 2-10 minutes. The samples were then placed on ice for 10 minutes followed by addition of 10 ul of anti-rat-CD11b-FITC or FITC-labeled mouse IgG2a isotype control (both Antigenix America, Huntington Station, N.Y.) and incubated for 30 minutes on ice. 1-2 ml of 1× FACS Lysing Solution (Becton Dickinson, San Jose, Calif.) was added with immediate vigorous vortexing, followed by additional vortex after the solution was added to the last sample. Samples were then incubated for 10 minutes at room temperature. The leukocytes were then pelleted at ˜300×g and washed with DPBS. Cells were then resuspended in 650 ul of 1% paraformaldehyde or 300 uL of Cell Fix (0.25% paraformaldehyde, 0.025 M sodium azide, DPBS and FACS flow). The FACS Lyse solution does not completely lyse rat red blood cells. Therefore, for flow cytometric analysis, LDS-751 was added to each sample within 5 minutes of analysis to enable elimination of red blood cells. Either 5 ul of a 0.02% methanol stock (Molecular Probes) or 3 ul of a 1.67 mg/ml ethanol stock (Exciton) was used. Samples were analyzed using an LSR or CANTOS II flow cytometer (Becton-Dickinson) gating on the neutrophil population in the side scatter versus forward scatter plot. FL1 (green FITC fluorescence, directly relating to CD11b content) of this population was then measured as mean channel fluorescence.

The CXCL2 dose response for Example 1 and Example 2 was evaluated. In both cases there was a rightward shift of the mean EC50 value (vehicle 4.6 nM; Example 2=12.3; Example 1=17.2). The ED50 for the dose response of CXCL2 was not significantly different between the compound from Example 1 or Example 2. In addition, the CXCL2 dose response was evaluated at several oral doses of the hydrochloride salt and compared with the inhibition of pulmonary neutrophilia in response to LPS-stimulation Inhibition of CD11b upregulation followed a similar dose response to that of inhibition of neutrophilia, with inhibition from 22% or 13% at 1 mg/kg, orally (p.o.) of neutrophilia or maximal CD11b expression to 96.4% or 65.7% at 30 mg/kg of neutrophilia or maximal CD11b expression.

### b) Cigarette Mmoke Exposure of Mice and Bronchoalveolar Lavage (BAL) Cell Counting:

Mice (Balb/c) were dosed orally with 15 or 30 mg/kg of the compound of Example 1, volume of 10 ml/kg, or vehicle 1 hour prior to the first cigarette and immediately after the last daily cigarette on each of the 3 days of smoke exposure. Mice were placed into a small plexiglass chamber, 6 animals at a time. The chamber was fitted with an intake and outflow port. A cigarette (Reference 4A1, University of Kentucky Tobacco Institute) was attached to the intake of a peristaltic pump, and the smoke was driven into the intake port of the chamber containing the mice. An air-flow (250 ml/min) was also delivered into the chamber and mixed with the cigarette smoke. The mice were exposed to the cigarette smoke for 4 minutes, the time required for the cigarette to burn down. Mice were exposed to 3 cigarettes per day for 3 days, with a minimum time of 2 hours between cigarettes. On day 4, mice were euthanized with Fatal Plus (i.p.) and bronchoalveolar lavaged with phosphate-buffered saline (5×0.7 ml). Cells were spun down, total cell counts were performed and slides were made on a cytospin and stained with Diff-Quick for differential cell counts. Cell counting was performed by counting 200 cells in cytospin samples by standard microscopy techniques. The percentage number of cells present was calculated and the total number of macrophages and neutrophils, in the BAL sample was then determined.

Cigarette smoke exposure induced a significant recruitment of neutrophils in BAL fluid compared to untreated control mice. Oral treatment with the compound of Example 1 caused an inhibition of neutrophils by 56% at 15 and 30 mg/kg doses. Exposure of mice to 3 cigarettes per day for 3 days resulted in neutrophil recruitment into the lungs as measured by BAL approximately 18 hours after the last cigarette. There was no significant increase in any other cell types detected in BAL fluid after cigarette smoke exposure including macrophages, lymphocytes and eosinophils. In vehicle-treated mice, cigarette smoke caused an increase in BAL neutrophils with a mean of 22.8±8.9×10⁴ cells compared to 0.3±0.2×10⁴ in naive, unexposed mice (p<0.05). Oral pretreatment of smoke-exposed mice with the compound of Example 1 (15 or 30 mg/kg) caused an inhibition in neutrophils detected in the BAL fluid. Both doses exhibited 56% inhibition of neutrophils recruited into the BAL fluid compared to the vehicle-treated group, although neither difference attained statistical significance.

### c) Ozone-Induced Pulmonary Neutrophilia in Mice or Rats:

Balb/c mice were dosed orally with the compound of Example 1, one hour prior to ozone exposure. Mice were exposed to ozone (3 parts/million, ppm) or filtered air for 6 hrs, and BAL were performed at 24 post completion of the ozone exposure. Lewis rats were dosed orally with Example 1 1 hr prior to ozone exposure (3 ppm, 3 hrs), and BAL were performed at 4 hr post ozone exposure. In both cases, cytospins were obtained followed by differential cell staining using May-Grunwald and Giemsa solutions. Cell counting was performed by counting 300 cells, the percent of differential cells was calculated, and the total number of neutrophils, in the BAL was determined based on total cell counts.

In mice, ozone caused an increase in BAL neutrophils, for example, from 0.07×10⁵±0.05 cells in air-exposed mice to 3.72×10⁵+1.03 in ozone exposed mice. Treatment with the compound of Example 1 caused a dose dependent decrease in neutrophils (70% inhibition at 30 mg/kg, p.o.; 36% inhibition 10 mg/kg, and 6% @ 3 mg/kg). Only the inhibition at 30 mg/kg was statistically significant using a one-way ANOVA with Bonferroni's correction, to p<0.05.

In rats, ozone exposure also resulted in an increase neutrophils in the BAL, for example from 1.8×105=/−0.05 cell to 11.8×105=/−1.1.cells. Treatment with the compound of Example 1 demonstrated dose dependent inhibition of neutrophils (12% @ 10 mg/kg; 57% @ 50 mg/kg).

### Human Studies in Healthy Subjects:

The inhibition of CXCL 1-induced CD11b expression following a single dose of the compound of Example 2 to human volunteers, using similar methodology as the rat CXCL2-induced CD11b expression, with some variations. For example, no lysis of red blood cells was included, and were excluded from the flow cytometry analysis by gating as in the rat studies. The CXCL1 dose response was determined between 1 and 1000 nM. As in the studies in rat, a clear dose-response effect was obtained at doses of the compound of Example 2 from 1 mg to 1100 mg. Inhibition was sustained for over 24 hrs at doses greater than or equal to 150 mg.

### Examples

Nuclear magnetic resonance spectra were recorded at either 300 or 400 MHz using, respectively, a Bruker ARX 300 or Bruker AVANCE 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (Δ) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Fourier transform infrared (FTIR) spectra were recorded on a Nicolet 510 infrared spectrometer. FTIR spectra were recorded in transmission mode, and band positions are reported in inverse wave numbers (cm⁻¹). Mass spectra were taken on either a SCIEX5 or Micromass instruments, using electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel. Analytical and preparative HPLC were carried out on Rainin or Beckman chromatographs. ODS refers to an octadecylsilyl derivatized silica gel chromatographic support. 5μ, Apex-ODS indicates an octadecylsilyl derivatized silica gel chromatographic support having a nominal particle size of 5μ, made by Jones Chromatography, Littleton, Colo. YMC ODS-AQ® is an ODS chromatographic support and is a registered trademark of YMC Co. Ltd., Kyoto, Japan. PRP-1® is a polymeric (styrene-divinylbenzene) chromatographic support, and is a registered trademark of Hamilton Co., Reno, Nev.) Celite® is a filter aid composed of acid-washed diatomaceous silica, and is a registered trademark of Manville Corp., Denver, Colo.

The following Examples are intended to be illustrative only and not limiting in any way.

### Example 1

### N-[4-chloro-2-hydroxy-3-(piperazine-1-sulfonyl)phenyl]-N′-(2-chloro-3-fluorophenyl)urea hydrochloride

### 1a) 2-chloro-3-fluorobenzoic acid

A solution of 3-fluorobenzoic acid (4.02 g, 28.71 mmol) in 20 mL of THF was added dropwise to a suspension of tetramethylenediamine (TMEDA) (10.00 mL, 66.3 mmol) and 1.3M sec-BuLi (48 mL, 62.4 mmol) in 50 mL of THF at −90° C. The mixture was stirred at −90° C. for 35 min. The mixture was warmed to −78° C. when a solution of hexachloroethane (27.0 g, 113.9 mmol) in 50 mL of THF was added. After 20 h, the reaction was quenched with water and diluted with diethyl ether. The bilayer was adjusted to pH ˜1-2 with conc. hydrochloric acid (HCl). The organic layer was washed with water, brine, dried and concentrated to give 30.4 g crude as a tan solid, which was washed with hexane to give 3.728 g (74%) of the desired product 1a (light tan solid). MS (m/z) 175.2 (M+H).

### 1b) 3-chloro-2-fluoro-benzoyl azide

A suspension of 2-chloro-3-fluorobenzoic acid (2.704 g, 15.54 mmol) in 25 mL of oxalyl chloride was heated to reflux for 2 hours. The solution was cooled and concentrated to give the crude acid chloride 3.13 g as a brown liquid which was directly used in the next step.

A solution of NaN₃ (2.79 g, 43 mmol) in 10 mL of water was added dropwise to a solution of the crude acid chloride (3.13 g) in 20 mL acetone at 0° C. After 15 min, the solution was diluted with CH₂Cl₂ and washed with water and brine. The organic layer was dried and concentrated to give a brown liquid which was filtered through silica gel using ethyl acetate/hexane (5/95, v/v) to yield 2.97 g (96%) of 1b (colorless liquid). The compound was used without further purification.

### 1c) 2-(2-tert-Butyl-6-chloro-benzooxazole-7-sulfonyl)-piperazine-1-carboxylic acid tert-butyl ester

The solution of 2-tert-butyl-6-chloro-benzooxazole-7-sulfonyl chloride (10.75 g, 34.9 mmol) in 100 mL of THF was cooled to 0° C., Et₃N (3.47 mL, 24.9 mmol) and then Boc-piperazine (5.0 g, 26.8 mmol) were added. The resulting mixture was stirred for 20 h, warming to room temperature. The mixture was poured into water, extracted with EtOAc and washed with another portion of water; organic layers were dried and concentrated. Purification by column chromatography on silica gel, eluting with ethyl acetate/hexane (30/70, v/v), yielded 11.12 g (91%) of the titled product. LC-MS (m/z) 458.2 (M+H).

### 1d) 4-(3-Amino-6-chloro-2-hydroxy-benzenesulfonyl)-piperazine-1-carboxylic acid tert-butyl ester

The solution of starting material 1e (11.12 g) in dioxane (20 mL) was treated with water (11 mL) and concentrated H₂SO₄ (11 mL). The mixture was heated to reflux for 12 h. The reaction mixture was concentrated and then basified the residue to pH ˜14 with 50% aq NaOH. (Boc)₂O (5.6 g, 1.05 eq) and 100 mL of AcOEt were added, the resulting mixture was stirred at room temperature for 16 h. The mixture was separated, the water layer was extracted with EtOAc, organic layers were combined, dried and concentrated. Purification by column chromatography on silica gel, eluting with ethyl acetate/hexane (30/70, v/v), yielded 8.18 g (85%) desired product 1d. ¹H NMR (CDCl3): δ 6.85 (m, 2H), 3.48 (t, 4H), 3.25 (t, 4H), 1.47 (s, 9H).

### 1e) 4-(6-chloro-3-[3-(2-chloro-3-fluorophenyl)-ureido]-2-hydroxy-benzenesulfonyl)-piperazine-1-carboxylic acid tert-butyl ester

A solution of 4-(3-amino-6-chloro-2-hydroxy-benzenesulfonyl)-piperazine-1-carboxylic acid tert-butyl ester (3.8 g, 9.7 mmol) and 3-chloro-2-fluorobenzoyl azide (2.9 g, 14.5 mmol) in 5 mL of N,N-dimethylformamide was stirred at room temperature for 18 h. The mixture was diluted with ethyl acetate and washed with water to give the crude material. Purification by column chromatography on silica gel, eluting with ethyl acetate/hexane (20/80, v/v), gave 3.6 g (66%) of 1e. LC-MS (m/z) 562.8 (M+H).

### 1f) N-[4-chloro-2-hydroxy-3-(piperazine-1-sulfonyl)-phenyl]-N′-(2-chloro-3-fluorophenyl)urea hydrochloride

A solution of 3.6 g Boc-product (1 g) in 20 mL of 4N HCl in dioxane was stirred at room temperature for 2 h and the solvent was evaporated. The residue was recrystallized from methanol and ethyl acetate to give the title product 2.9 g (60%). LC-MS (m/z) 463.0 (M+H).

### Example 2

### N-[4-chloro-2-hydroxy-3-(piperazine-1-sulfonyl)phenyl]-N′-(2-chloro-3-fluorophenyl)urea p-toluenesulfonate

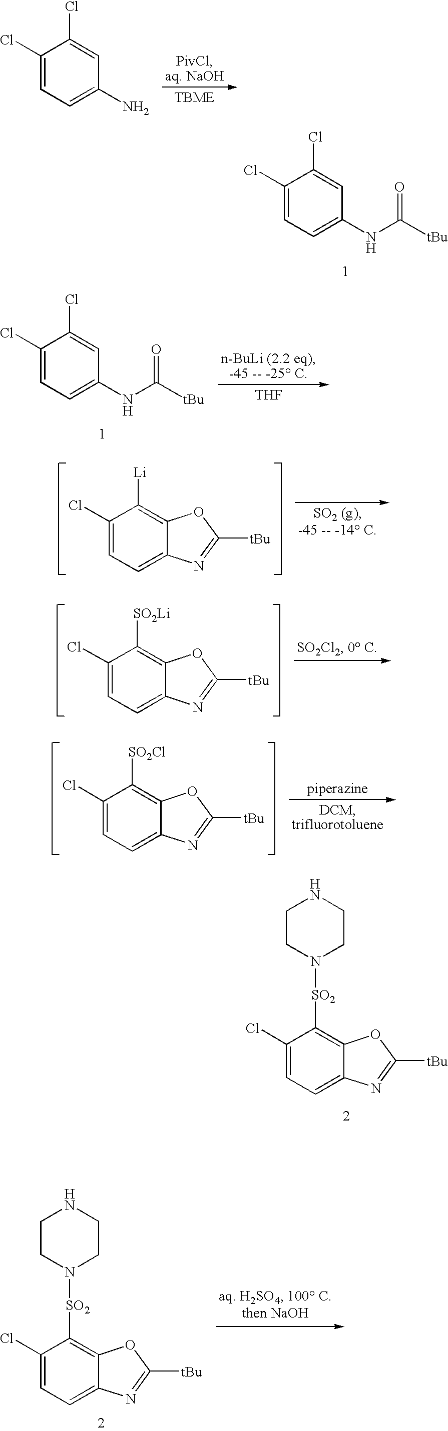

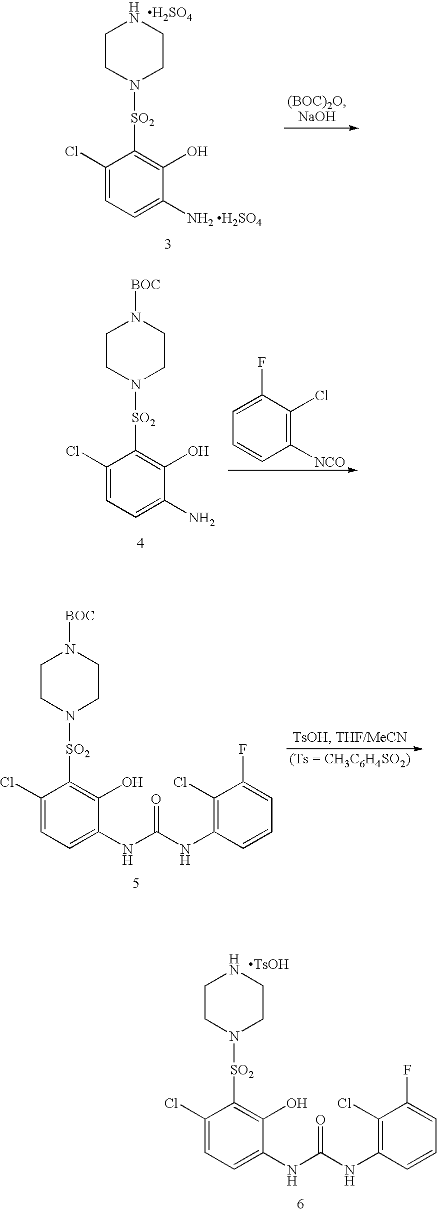

### 2a) Preparation of Compound 1 in Accordance with the Scheme Above

3,4-dichloroaniline (100 g) was dissolved in TBME (660 mL) and cooled to 10-15° C. Sodium hydroxide (94 g of a 30% aqueous solution) was added, and the solution stirred vigorously via mechanical stirrer. Trimethylacetyl chloride (84 mL) was added at such a rate as to keep the internal temperature below 35° C. When the addition was complete (10-15 min), the mixture was maintained at 30-35° C. for about 30 min, and then cooled to 0-5° C. over 30-40 minutes. The reaction mixture was held at 0-5° C. for 1 hour, and then filtered, rinsing first with 90:10 water/methanol (400 mL) and then water (600 mL.) Drying at 50-55° C. under vacuum afforded product as off-white crystals. A yield of 127 g was obtained.

### 2b) Preparation of Compound 2

A solution of Compound 1 (50 g) in tetrahydrofuran (300 mL) was cooled to −50-−40° C. under an inert atmosphere of nitrogen. N-Butyl lithium (2.5M in hexanes, 179 mL) was added at such a rate as to keep the solution's internal temperature between −45-−30° C. (ca. 15-30 min addition). The solution was held at ca. −35-−25° C. until HPLC indicated that the initial reaction was complete. The solution was then recooled to −45-−40° C., and sulfur dioxide (˜16.9 g) was bubbled through the solution, keeping the internal temperature below approximately −14° C., until the solution was acidic. When the reaction was complete, the mixture was warmed to −10-0° C. Starting at −2-3° C., sulfuryl chloride (25.2 mL) was then added dropwise to the tetrahydrofuran solution over 5-15 min, keeping the temperature below approximately 22° C. After 5 min, HPLC confirmed reaction completion, while the solution was kept around 10-15° C. The mixture was solvent-exchanged into a,a,a-trifluorotoluene under reduced pressure, filtered, partially concentrated under vacuum (to ˜100 mL), followed by addition of dichloromethane (350 mL). To this mixture was added a solution of piperazine (61.2 g) in dichloromethane (625 mL) at ambient temperature dropwise, keeping the solution's internal temperature at 15-27° C. (2 h addition). The reaction was held at 20-24° C. until complete. The mixture was washed with deionized water (200 mL), the organic layer concentrated, followed by addition of heptane (450 mL). The product (70.5 g) was isolated by filtration, washed with heptane (50-100 mL), and dried under vacuum at 50-55° C.

### 2c) Preparation of Compound 3

Compound 2 (30 g) was added to ˜16% (w/w in water) sulfuric acid (300 mL). The resulting mixture was heated to reflux at 99-103° C. for ˜6 hours. Upon completion of the reaction, the solution was cooled to 40-50° C., then concentrated to ˜60 mL under reduced pressure. Acetonitrile (225 mL) was added and the resulting suspension stirred at 20-25° C. for ˜1 hour. The product was isolated by filtration, washed with acetonitrile (135 mL) and dried at 45-50° C. under vacuum. A yield of 33.34 g was obtained.

### 2d) Preparation of Compound 4

Compound 3 (20 g) was added to deionized water (200 mL). The pH of the resulting solution was adjusted to 6.5-7.0 by adding 50% aq. sodium hydroxide (˜6.35 mL) while maintaining the internal temperature between 20-30° C. Then a solution of di-tent-butyl dicarbonate (8.9 g) in ethyl acetate (80 mL+20 mL rinse) was added. The pH of the resulting mixture was adjusted to 6.8-7.0 by adding 50% aq. sodium hydroxide (2.45 mL) while maintaining the internal temperature between 20-30° C. Upon completion of the reaction, the reaction solution is filtered to remove the small amount of precipitate. The two layers of the filtrate were separated, and the aqueous layer was extracted with ethyl acetate (140 mL). Combined ethyl acetate layers are washed with water (40 mL) and concentrated to 100 mL. Heptane (100 mL) was added and the resulting suspension was concentrated to 60 mL. This process was repeated once more. Heptane (140 mL) was then added, and the resulting suspension was stirred at 20-25° C. for ˜1 hour. The product was isolated by filtration, washed with heptane (80 mL) and dried at 40-45° C. under vacuum. A yield of 15.3 g was obtained.

### 2e) Preparation of Compound 5

Compound 4 (10 g) was added to dimethylformamide (20 mL) and acetonitrile (80 mL). 2-Chloro-3-fluorophenyl isocyanate (4.77 g) was added while maintaining the internal temperature between 20-30° C., followed by 10 mL acetonitrile rinse. The resulting mixture was stirred at 20-25° C. for ˜2 hours. Upon completion of the reaction, methanol (50 mL) was added. The resulting suspension was stirred at 20-25° C. for ˜10 minutes. Deionized water (150 mL) was added, and the resulting suspension stirred at 20-25° C. for ˜1 hour. The product was isolated by filtration, washed with deionized water (100 mL) and methanol (15-20 mL), and then dried at 40-45° C. under vacuum. A yield of 14.15 g was obtained.

### 2f) Preparation of Compound 6—Procedure 1

Compound 5 (50 g) was dissolved in tetrahydrofuran (THF, 200 mL) and heated to 33-37° C. and held at 33-37° C. In another reactor, a solution of acetonitrile (250 mL), THF (50 mL) and p-toluenesulfonic acid monohydrate (43.9 g) was prepared. The resulting solution was heated to 33-37° C. and held at 33-37° C. The p-toluenesulfonic acid solution was filtered and transferred into the reactor containing Compound 5 and THF while maintaining the temperature at 33-37° C. After the starting material was consumed, micronized seeds of product (0.5 g) were charged in a minimal amount of acetonitrile (5 mL). The reaction mixture was then heated to 53-57° C. over ˜40 minutes, and held at that temperature for at least 4 hours. The reaction was cooled to 0-5° C., the product isolated by filtration, washed with acetonitrile (250 mL), and dried under vacuum at 55-60° C. A yield of 52.24 g was obtained.

### 2f) Preparation of Compound 6—Procedure 2

Compound 5 (500 g) was charged to reactor 1 followed by acetonitrile (CAN, 3750 mL) and tetrahydrofuran (THF, 1250 mL). The solution was then heated to 60-65° C. and once a clear solution is observed, a clarifying filtration is performed to reactor 2. To reactor 1, p-toluenesulfonic acid monohydrate (TsOH.H₂O, 439 g) is added followed by ACN (750 mL) and THF (250 mL). The mixture was heated to 40-45° C. and once a clear solution was observed, a clarifying filtration was performed, adding the solution to reactor 2 (containing the starting material solution) and maintaining the temperature in reactor 2 at 50-60° C. The mixture was heated to reflux, and held at 70-80° C. until the reaction was complete. ˜3500 mL of solvent was removed by atmospheric distillation. The reactor was then charged with 2.5 L water followed by 4 L ACN, and the temperature adjusted to 70-80° C. After dissolution was observed, the resulting solution was cooled to 64-68° C. After 5-10 minutes, milled product Form III seeds (5 g) were added in a minimal amount of acetonitrile, and held at 64-68° C. for one hour. The mixture was cooled to 0-5° C. over 2 hours and held at 0-5° C. for ˜30 minutes before isolating the product by filtration. The solid product was washed with 2.5L of acetonitrile, and dried under vacuum at 50-60° C. A yield of 480 g was obtained.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

What is claimed is:

1. A method of treating cystic fibrosis in a human in need thereof, comprising administering to said human an effective amount of N-[4-chloro-2-hydroxy-3-(piperazine-1-sulfonyl)phenyl]-N′-(2-chloro-3-fluorophenyl)urea or a pharmaceutically acceptable salt thereof.

2. The method according to claim 1 wherein the compound is N-[4-chloro-2-hydroxy-3-(piperazine-1-sulfonyl)phenyl]-N′-(2-chloro-3-fluorophenyl)urea hydrochloride.

3. The method according to claim 1 wherein the compound is N-[4-chloro-2-hydroxy-3-(piperazine-1-sulfonyl)phenyl]-N′-(2-chloro-3-fluorophenyl)urea p-toluenesulfonate.

4. The method according to claim 1 wherein the compound is N-[4-chloro-2-hydroxy-3-(piperazine-1-sulfonyl)phenyl]-N′-(2-chloro-3-fluorophenyl)urea.

5. The method according to claim 1 wherein the compound is administered orally.

6. A method of slowing the progression of cystic fibrosis in a human in need thereof, comprising administering to said human an effective amount of a compound which is N-[4-chloro-2-hydroxy-3-(piperazine-1-sulfonyl)phenyl]-N′-(2-chloro-3-fluorophenyl)urea or a pharmaceutically acceptable salt thereof.

7. The method according to claim 6 wherein the compound is N-[4-chloro-2-hydroxy-3-(piperazine-1-sulfonyl)phenyl]-N′-(2-chloro-3-fluorophenyl)urea hydrochloride.

8. The method according to claim 6 wherein the compound is N-[4-chloro-2-hydroxy-3-(piperazine-1-sulfonyl)phenyl]-N′-(2-chloro-3-fluorophenyl)urea p-toluenesulfonate.

9. The method according to claim 6 wherein the compound is N-[4-chloro-2-hydroxy-3-(piperazine-1-sulfonyl)phenyl]-N′-(2-chloro-3-fluorophenyl)urea.

10. The method according to claim 6 wherein the compound is administered orally.

11. A method of alleviating the symptoms of cystic fibrosis in a human in need thereof, comprising administering to said human an effective amount of N-[4-chloro-2-hydroxy-3-(piperazine-1-sulfonyl)phenyl]-N′-(2-chloro-3-fluorophenyl)urea or a pharmaceutically acceptable salt thereof.

12. The method according to claim 11 wherein the compound is N-[4-chloro-2-hydroxy-3-(piperazine-1-sulfonyl)phenyl]-N′-(2-chloro-3-fluorophenyl)urea hydrochloride.

13. The method according to claim 11 wherein the compound is N-[4-chloro-2-hydroxy-3-(piperazine-1-sulfonyl)phenyl]-N′-(2-chloro-3-fluorophenyl)urea p-toluenesulfonate.

14. The method according to claim 11 wherein the compound is N-[4-chloro-2-hydroxy-3-(piperazine-1-sulfonyl)phenyl]-N′-(2-chloro-3-fluorophenyl)urea.

15. The method according to claim 11 wherein the compound is administered orally.
